(19) United States
(12) Patent Application Publication
(10) Pub. No.: US 2016/0091445 A1
(43) Pub. Date: Mar. 31, 2016

(54) Hydrogen Gas Sensor And Method For Fabrication Thereof

(71) Applicant: UNITED ARAB EMIRATES UNIVERSITY, Al Ain (AE)

(72) Inventor: Ahmad Ibrahim Ayesh, Al Ain (AE)

(21) Appl. No.: 14/888,368

(22) PCT Filed: Jun. 9, 2014
(86) PCT No.: PCT/IB2014/062080

§ 371 Date: Oct. 30, 2015

Related U.S. Application Data
(60) Provisional application No. 61/833,083, filed on Jun. 10, 2013.

(51) Int. Cl. G01N 27/02; C23C 14/16; G01N 33/00

(52) U.S. Cl. CPC G01N 27/02; G01N 33/005; C23C 14/165

(57) ABSTRACT

A hydrogen gas sensor and a method for fabrication thereof are disclosed. The hydrogen gas sensor includes an insulating substrate, a pair of electrical electrodes deposited thereon, and a nanocluster film formed intermediate said electrical electrodes such that hydrogen concentration in ambient air surround the hydrogen gas sensor is measurable based on a change in electrical current established through the nanocluster film using a constant voltage power supply.

### BACKGROUND OF THE PRESENT INVENTION

1. Technical Field

The present invention relates to gas sensors, and more particularly, to a hydrogen gas sensor and a method of fabrication thereof.

2. Description of the Related Art

Hydrogen is emerging as an important source of clean energy, and offers several advantages as a clean fuel. The potentially unlimited supply of hydrogen in nature and pollution-free combustion are compelling reasons for adoption of hydrogen as a fuel.

Owing to high combustibility of hydrogen, one of the pre-requisites for adoption for hydrogen-based clean energy technologies is reliable hydrogen sensing modalities in order to ensure safety and prevent loss of man and materials arising from undetected hydrogen leakage.

In recent years, several different sensing modalities have been proposed for sensing hydrogen gas. Among different types of hydrogen sensors available in the state of the art, electrical conductivity based hydrogen sensors appear to be most promising. One such example is palladium-based hydrogen sensors that consist of macroscopic/microscopic palladium structure.

Such state of the art hydrogen sensors suffer from several drawbacks.

The state of the art sensors are based on the principle that the conductivity of palladium crystals decreases upon exposure to hydrogen gas relative to the unexposed palladium. Accordingly, such sensors measure hydrogen concentration as an inverse relationship to conductivity of palladium. Such state of the art sensors do not provide a linear relation between concentration of hydrogen in ambient air and change in conductivity. Accordingly, it is difficult to calibrate such state of the art sensors.

A further disadvantage of state of the art sensors is relatively fast saturation and undesirably low range of operation. Owing to high affinity of palladium towards hydrogen, the palladium crystals readily become saturated upon exposure to even small amounts of hydrogen and thereby, such devices are rendered useless if higher concentrations of hydrogen are to be measured. Moreover, the state of the art hydrogen sensors require heating to release the hydrogen adsorbed on palladium crystals and revive the sensor for next measurement. Evidently, such sensors not only consume high power but also increase risk of explosions.

Such sensors lack desired selectivity. The palladium used in such sensors is prone to combination with such other gases as sulphur-dioxide, methane, and so on, and presence of such gases even in trace amounts is sufficient to severely impact hydrogen sensing ability of palladium-based hydrogen sensors due to blocking of adsorption sites therein.

Yet another disadvantage of state of the art sensors is that the response time is undesirably high, requiring up to several minutes for detecting hydrogen.

In light of the foregoing, there is a need for a hydrogen sensor with simple calibration, enhanced range of operation, reduced power consumption, high selectivity, and reduced response time.

### SUMMARY OF THE PRESENT INVENTION

It is an object of the present invention to provide a hydrogen sensor exhibiting a linear relationship between a measured parameter and concentration of hydrogen gas in ambient air.

It is another object of the present invention to provide a hydrogen sensor with enhanced range of operation.

It is still another object of the present invention to provide a hydrogen sensor with reduced power consumption.

It is another object of the present invention to provide a hydrogen sensor with high selectivity.

It is another object of the present invention to provide a hydrogen sensor with reduced response time.

It is yet another object of the present invention to provide a method for fabrication of such hydrogen sensor.

The object is achieved by providing a hydrogen sensor according to claim 1 and a method for fabricating the same according to claim 7. Further embodiments of the present invention are addressed in respective dependent claims.

The underlying concept of the present invention is to fabricate a hydrogen gas sensor based on changes in electrical conductivity of a nanocluster film formed using inert-gas condensation techniques such that palladium constitutes about 77(+/−1) percent and copper constitutes about 23(+/−1) percent of the nanocluster film. The nanocluster film formed in accordance with the disclosed method provides desirable properties related to electrical and adsorption characteristics of the nanocluster film.

In a first aspect of the present invention, a method for fabricating a hydrogen gas sensor is provided. At a first step, an insulating substrate is provided and a pair of electrical electrodes is deposited thereon. Subsequently, nanoclusters of palladium-copper are generated using sputtering and inert-gas condensation techniques such that palladium percentage ranges from about 76 percent to about 78 percent and copper percentage ranges from about 22 percent to about 24 percent. Finally, a nanocluster film is deposited intermediate said electrical electrodes, wherein said nanocluster film comprises said nanoclusters of palladium-copper alloy.

In a second aspect of the present invention, a hydrogen gas sensor, as described in accordance with the first aspect of the present invention, is provided. The hydrogen gas sensor comprises an insulating substrate, a pair of electrical electrodes deposited thereon, and a nanocluster film intermediate said electrical electrodes, wherein said nanocluster film comprises nanoclusters of palladium-copper generated using sputtering and inert-gas condensation techniques such that palladium ranges from about 76 percent to about 78 percent and copper ranges from about 22 percent to about 24 percent.

The present invention provides a hydrogen gas sensor and a method for fabrication thereof such that calibration is simplified, range of operation is enhanced, power consumption is reduced, selectivity towards adsorption of hydrogen is increased, and response time to detect hydrogen concentration is reduced.

### BRIEF DESCRIPTION OF THE DRAWINGS

The present invention is further described hereinafter with reference to illustrated embodiments shown in the accompanying drawings, in which:

FIG. 1 illustrates a schematic view of a hydrogen gas sensor in accordance with the present invention,

FIG. 2 illustrates size distribution of palladium-copper nanoclusters in accordance with the present invention,

FIG. 3 illustrates variation of electrical current through the nanocluster film during fabrication in accordance with the present invention,

FIGS. 4A-4B illustrate variation of response signal during measurement of progressively increasing concentrations of hydrogen gas in ambient air in accordance with the present invention,

FIG. 5 illustrates variation of response signal during measurement in a given hydrogen gas sensor on repeated exposure to same hydrogen concentration in accordance with the present invention,

FIG. 6 illustrates variation of response signal during measurement in different hydrogen gas sensors on exposure to different hydrogen concentrations in accordance with the present invention,

FIG. 7 illustrates variation of response time in different hydrogen gas sensors on exposure to different hydrogen concentrations in accordance with the present invention, and

FIG. 8 illustrates a method for fabrication of a hydrogen gas sensor in accordance with the present invention.

### DETAILED DESCRIPTION OF THE PRESENT INVENTION

Various embodiments are described with reference to the drawings, wherein like reference numerals are used to refer to like elements throughout. In the following description, for purpose of explanation, numerous specific details are set forth in order to provide a thorough understanding of one or more embodiments. It may be evident that such embodiments may be practised without these specific details.

The present invention relates to a hydrogen gas sensor and a method for fabrication thereof. The hydrogen gas sensor of the present invention is based on use of a nanocluster film of palladium-copper nanoclusters adapted to be substantially near its percolation threshold.

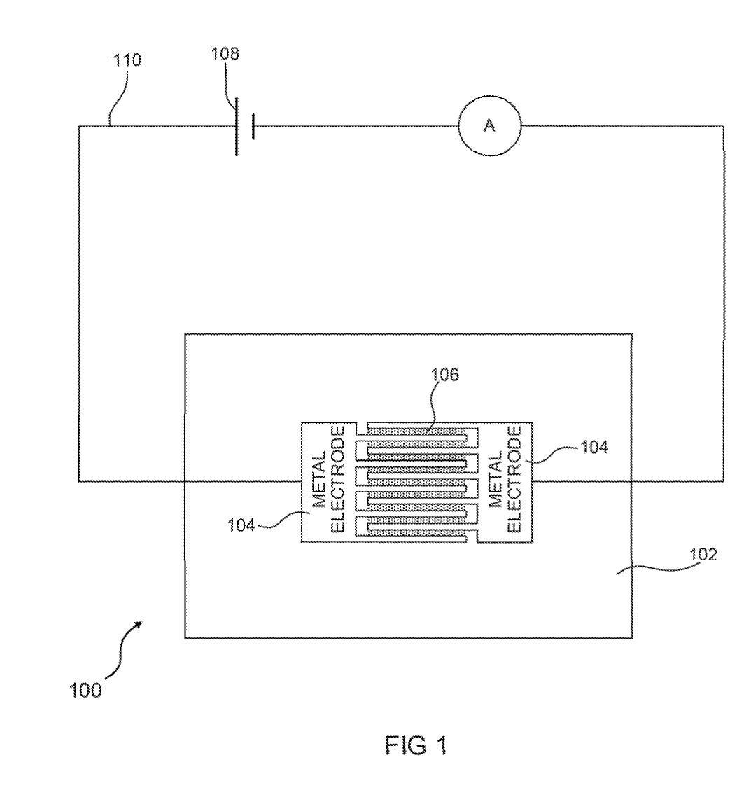

Referring now to FIG. 1, a schematic view of a hydrogen gas sensor 100 in accordance with the present invention is illustrated.

The hydrogen gas sensor 100 includes an insulating substrate 102 and a pair of electrical electrodes 104 deposited thereon. The hydrogen gas sensor 100 further includes a nanocluster film 106 intermediate the electrical electrodes 104. Further depicted in FIG. 1 are a power supply 108 and electrical interconnections 110. It should be noted that while the power supply 108 and the electrical interconnections 110 may be included in the hydrogen gas sensor 100, these components are not contemplated to be integral to the hydrogen gas sensor 100 and may be externally connected during operation.

In accordance with the fabrication process, initially an insulating substrate 102 such as glass is provided and a pair of electrical electrodes 104 is formed thereon in the form of thin metal strips. In a preferred embodiment of the present invention, the thin metal strips are formed of two layers of gold on nichrome using a shadow mask technique.

In one example, the insulating substrate 102 is 10 mm×10 mm, while the pair of electrical electrodes 104 spans over a 5 mm×5 mm area on the insulating substrate 102. It should be noted that these dimensions are absolutely exemplary in nature and any suitable dimensions may be used for fabricating the hydrogen gas sensor 100 of the present invention.

In an exemplary embodiment of the present invention, the electrical electrodes 104 are formed as interdigitated structures such as to increase an interface surface thereof. As will become apparent from the following description, this technical feature facilitates increasing contact area between the electrical electrodes 104 and the nanocluster film 106, which helps improving signal-to-noise ratio in an electrical current established through the nanocluster film 106. If it is desired to further improve the signal-to-noise ratio, multiple pairs of the electrical electrodes 104 may be used and connected in parallel across the power supply 108.

In various exemplary embodiments of the present invention, the typical separation between the electrical electrodes 104 is in the range of 20 to 40 microns.

After formation of the electrical electrodes 104, the nanocluster film 106 is deposited intermediate the electrical electrodes 104. The nanocluster film 106 includes nanoclusters of palladium and copper. In the nanocluster film 106, palladium ranges from about 76 percent to about 78 percent and copper ranges from about 22 percent to about 24 percent. In accordance with various exemplary embodiments of the present invention, the nanoclusters of palladium and copper are generated using sputtering and inert-gas condensation techniques.

It should be noted that various palladium-copper nanocluster synthesis techniques are available in the prior art. One example of such technique is urea gelation and template-assisted method [E S. Bickford, S. Velu, C S. Song, Catalysis Today 99, 347 (2005)]. Another known technique is based on using a water-in-oil microemulsion system of water/dioctyl sulfosuccinate sodium salt (aerosol-OT, AOT)/isooctane at 25 degree C. Since the nanoclusters produced using this technique can endure relatively high temperatures (100 degree C.), this system is used for the synthesis of nano-catalysts in the Heck reactions [F. Heshmatpour, R. Abazari, S. Balalaie, Tetrahedron 68, 3001 (2012)]. In addition, a sacrificial support method in combination with chemical reduction of metal precursors can be used for the preparation of palladium-copper nanoclusters [A. Serov, U. Martinez, A. Falase, P. Atanassov, Electrochemistry Communications 22, 193 (2012)].

However, various such known techniques in the state of the art suffer from one or more drawbacks. First and foremost, owing to chemical synthesis commonly used in state of the art techniques, purity and control of relative percentages of individual metal nanoclusters is low. Further, control of nanocluster size as well as production of mono-dispersed nanoclusters is relatively difficult to achieve. Another important disadvantage is that the state of the art techniques do not enable self-assembly of nanoclusters directly on a target device in a controlled manner and hence, it is difficult to achieve precise control of thickness of nanocluster film subsequently formed on a target device.

In view of the foregoing shortcomings of bimetallic nanocluster synthesis techniques, the nanocluster film 106 of the present invention is formed using sputtering and inert-gas condensation technique. The inert-gas condensation technique has not been used before for fabrication of palladium-copper nanoclusters. The inert-gas condensation technique was adapted to precisely control relative percentages of individual metals within nanocluster film 106.

The nanocluster film synthesis system is similar to those described in “Size-controlled Pd nanocluster grown by plasma gas-condensation method” [A. I. Ayesh, S. Thaker, N. Qamhieh, and H. Ghamlouche, J. Nanopart. Res. 13, 1125 (2011)]; and “Fabrication of size-selected Pd nanoclustersusing a magnetron plasma sputtering source” [A. I. Ayesh, N. Qamhieh, H. Ghamlouche, S. Thaker, and M. E L-Shaer, J. Appl. Phys. 107, 034317 (2010)]. The nanocluster film synthesis system, as disclosed in these publications, was adapted to generate palladium-copper nanocluster film of the present invention.

For sake of completion, the nanocluster film synthesis system is being briefly described herein.

The nanocluster film synthesis system includes three chambers, namely, a nanocluster source chamber, a mass filter chamber, and a deposition chamber. The source and deposition chambers are pumped down to a pressure of about 10⁻⁶ mbar using two turbo pumps. Initially, metal vapor is produced inside the nanocluster source chamber. The metal vapor can be produced inside the source chamber by different methods such as: magnetron plasma sputtering (either AC or DC), thermal evaporation, arc discharge, electron beam heating, and laser irradiation. The nanocluster source chamber is provided with inert gas stream flowing over the source of metal vapor. The inert gas causes condensation of the metal vapor into small particles that is, nanoclusters. The inert gas stream carries the produced nanoclusters through a nozzle to the mass filter chamber that allows identifying the nanocluster mass/size and/or selecting nanoclusters of a required size. The nanoclusters leave the mass filter, forming a beam, to the deposition chamber, where the nanoclusters may be deposited on any suitable target surface.

Referring now to specific techniques of the present invention, nanocluster film of palladium-copper alloy is synthesized. Towards this end, a palladium target covered partially with a sheet of copper is used. The relative percentages of palladium and copper within the resulting nanocluster film 106 are regulated by controlling ratio of surface area of palladium target covered by copper. Given the relative percentages of palladium (77+/−1) and copper (23+/−1), as described earlier, and relative nanocluster yields of palladium-copper, the present nanoclusters were produced using a target with one-third of palladium surface area being covered with the sheet of copper.

The relative percentages of palladium and copper are essential to achieving desired adsorption properties (threshold of saturation on exposure of hydrogen and release of adsorbed hydrogen on removal of hydrogen from ambient air) and selectivity towards hydrogen (to eliminate adsorption of other gaseous species that may be present in the ambient air). These properties of the nanocluster film 106 further manifest in the form of a linear relationship between a response signal of the hydrogen gas sensor 100 and concentration of hydrogen in the ambient air.

Thus, for the specified relative percentages of palladium and copper, the hydrogen gas sensor 100 displays desired linear relationship between hydrogen concentration and resulting electrical current over an extended range of operation. As the relative percentages of palladium and copper deviate from the stipulated range, the linear characteristics the resulting hydrogen gas sensor is reduced.

It should be noted that relative percentages of palladium and copper, as disclosed in the present application, are critical to optimizing contradictory requirements related to other desired characteristics of the hydrogen gas sensor 100 such as enhanced range of operation, reduced power consumption, high selectivity, and reduced response time.

In particular, saturation threshold and power consumption are optimized for the stipulated percentage range of copper in the nanocluster film 106. If the relative percentage of copper is decreased, the nanocluster film 106 may become saturated at relatively lower concentration levels of hydrogen in ambient air and accordingly, the saturation threshold is reduced. This, in turn, reduces the range of operation of the resulting hydrogen gas sensor. On the other hand, if the relative percentage of copper is increased, the affinity of nanocluster film 106 towards hydrogen gas increases significantly. Accordingly, the nanocluster film 106 does not easily release hydrogen after a measurement operation is completed and hydrogen has been flushed from the ambient air. This, in turn, necessitates heating the nanocluster film 106 to release adsorbed hydrogen and hence, increases cost and complexity of manufacturing as well as that of operation of the resulting hydrogen sensor.

Referring back to the technique for fabrication of the nanocluster film 106, DC magnetron plasma sputtering is used to produce metal vapor of palladium and copper; argon inert-gas is used to produce plasma and effect condensation of the respective metal vapor.

The mass filter is regulated to select nanoclusters with an average diameter of about 8.1 nm. The target device, that is, the insulating substrate 102 with the electrical electrodes 104 formed thereon, is placed in the deposition chamber such that the nanocluster beam emanating from the mass filter is directed towards an area of insulating substrate 102 intermediate the electrical electrodes 104 and a nanocluster film 106 is formed thereon.

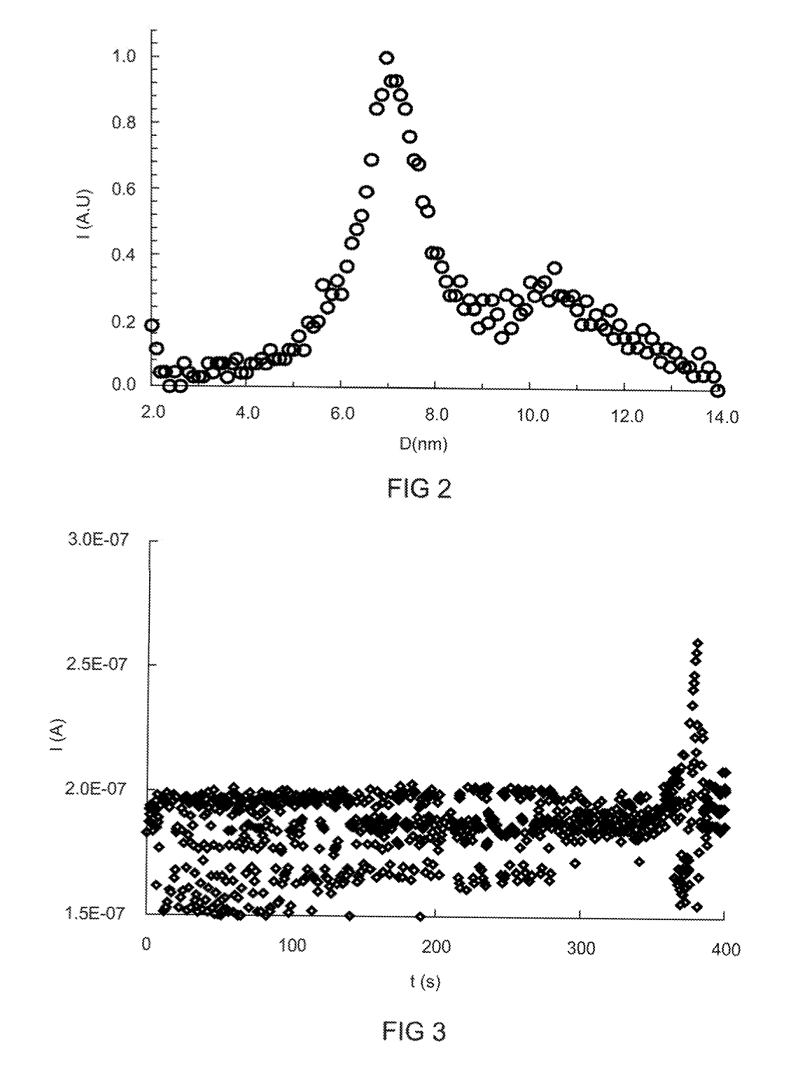

Referring now to FIG. 2, size distribution of palladium-copper nanoclusters is illustrated in accordance with an exemplary embodiment of the present invention. The size of nanoclusters may be measured using any suitable method such as quadruple mass filter, transmission electron microscope (TEM), and so on. As can be seen from the adjoining figure, the nanoclusters within the nanocluster film 106 have an average diameter in the range of 4 nm to 14 nm. As mentioned previously, the average size of the produced nanoclusters is 8.1 nm.

The dimension of palladium-copper nanoclusters further contributes to achieving desired adsorption properties (threshold of saturation on exposure of hydrogen and release of adsorbed hydrogen on removal of hydrogen from ambient air) and selectivity towards hydrogen (to eliminate adsorption of other gaseous species that may be present in the ambient air).

The formation and features of the nanocluster film 106 of the present invention will now be explained in detail.

Referring now to FIG. 3, variation of electrical current through the nanocluster film 106 during fabrication is illustrated in accordance with the present invention.

In accordance with techniques of the present invention, the nanocluster film 106 is configured to be substantially near a percolation threshold thereof such that the nanocluster film 106 is substantially non-conductive in absence of hydrogen and further such that the nanocluster film 106 is substantially conductive in presence of hydrogen.

The electrical conductivity of the nanocluster film 106 is linearly proportional to concentration of hydrogen in ambient air surrounding the nanocluster film 106.

As is generally well-understood in the art, a nanocluster film configured to be substantially near its percolation threshold includes a relatively small number of interconnected pathways and a relatively much larger number of isolated pathways between the nanoclusters. The electrical conduction is, therefore, attributable to combination of normal conduction through the interconnected pathways and conduction based on tunneling effect through the isolated pathways.

During fabrication process, prior to initiating deposition of nanocluster film 106, the electrical electrodes 104 are connected to the power supply 108 through the electrical interconnections 110 to establish a voltage gradient across the electrical electrodes 104.

During the deposition process, the electrical current established through said nanocluster film is monitored, as indicated in the adjoining figure. In particular, a signal-to-noise ratio of the electrical current is monitored. As will be understood that as the nanocluster film 106 approaches the corresponding percolation threshold, the electrical current there through will sharply increase and additionally, the signal-to-noise ratio will have a sharp reduction. Thus, according to techniques of the present invention, the indicative signal-to-noise ratio at which the percolation threshold is empirically measured and used during fabrication process to regulate the deposition process. It will be appreciated that given that form factor of insulating substrate 102 and electrical electrodes 104 and various process parameters remain unchanged, the empirical signal-to-noise ratio and electrical current values may be readily used to regulate the deposition such that the nanocluster film 106 with substantially near its percolation threshold is formed. Thus, during the fabrication process, deposition of nanoclusters is effected till a predefined signal-to-noise ratio is achieved in said electrical current.

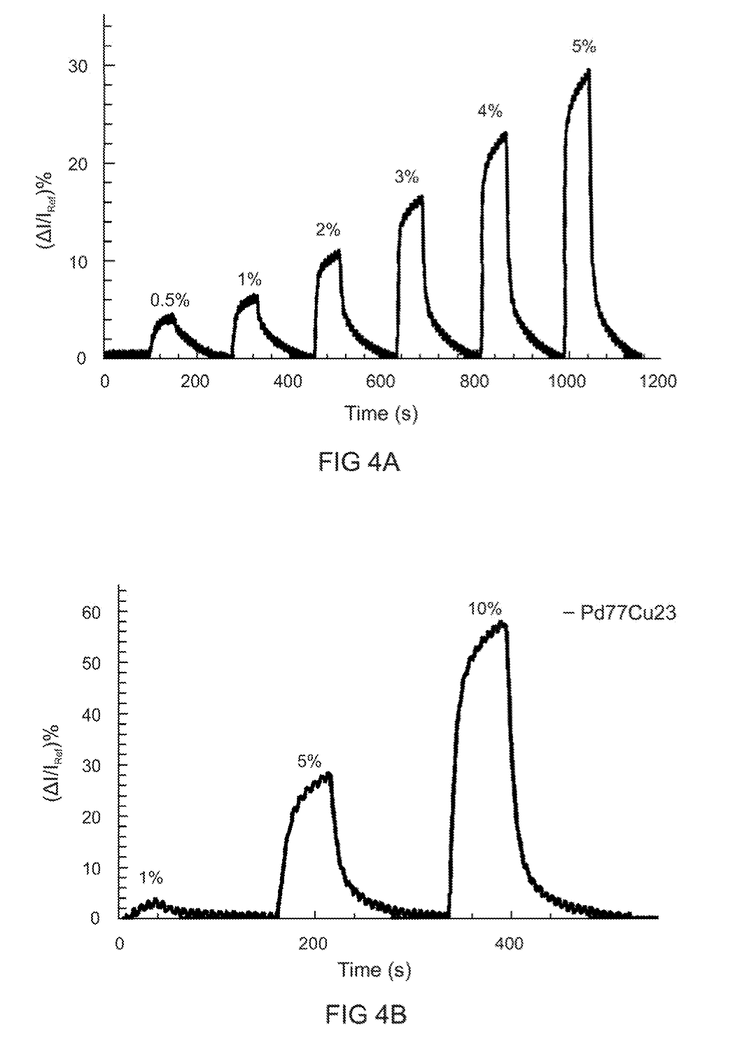

The operation of hydrogen gas sensor 100 will now be explained. The hydrogen gas sensor of the present invention may be operated by applying a voltage of about 100 mV across the inter-digitated electrical electrodes, and measuring the resulting electrical current through the nanocluster film 106 using a conventional ammeter, as shown in FIG. 1. In the context of measuring concentration of hydrogen in the ambient air, the electrical current through the nanocluster film 106 is hereinafter referred to as response signal. Further details of function and features of the hydrogen gas sensor 100 will now be explained in conjunction with FIGS. 4A and 4B.

Referring now to FIGS. 4A and 4B, variation of response signal through the nanocluster film during measurement of progressively increasing concentrations of hydrogen gas in ambient air is illustrated in accordance with an exemplary embodiment of the present invention.

As evident from the adjoining figure, the hydrogen gas sensor 100 provides relatively much higher sensitivity compared to various state of the art hydrogen gas sensors. As can be seen, when the hydrogen gas sensor 100 is exposed to a 0.5% hydrogen concentration in ambient air maintained at atmospheric pressure and 25 deg C, the figure of merit (ΔI/I), measured in terms of relative change in response signal (electrical current through the nanocluster film 106), is about 30%.

It should be noted that the measurement principle of the present invention is in contrast to that of various conventional hydrogen gas sensors based on microscopic palladium structure, where the response signal decreases upon exposure to hydrogen.

As is generally known in the art, exposing palladium to hydrogen causes the expansion of the face centered cubic (fcc) lattice by a maximum of 3.6% due to a phase change in the crystal structure, that is, from α to β phase. The phase expansion occurs along each nanocluster axis, and preferably at the grain boundaries. As a result, the inter-granular gaps of a palladium nanocluster film are reduced, thus, electrical conductance of the nanocluster film increases. However, the phase transition is manifested as a plateau in a plot of ambient hydrogen gas pressure versus hydrogen content of the palladium lattice at relatively small concentration levels of hydrogen in ambient air. When such a palladium nanocluster film is exposed to pure ambient air β to α phase transition ensues resulting in contraction of each nanocluster, thus, opening the gaps again within the nanocluster film causing the decrease in the electrical conductivity of the nanocluster film. However, this usually requires heating the palladium nanocluster film.

According to the techniques of the present invention, the nanocluster film 106 is formed using alloy of palladium and copper such that affinity of nanocluster film 106 towards hydrogen is regulated such as to inhibit fast saturation during measurement process and thereby, provide a higher operational range. Furthermore, the affinity of nanocluster film 106 is regulated in a manner to ensure that the adsorbed hydrogen gas is released at the end of measurement process without the need of heating of the nanocluster film 106 beyond the ambient temperature used during measurement. This technical feature of the present invention advantageously reduces power consumption of the hydrogen gas sensor 100.

The relative percentages of palladium and copper, as disclosed in the present disclosure, provide the nanocluster film 106 with the reduced affinity levels required to achieve the desired features of extended range of operation and reduced power consumption. As evident from the adjoining figure, the hydrogen gas sensor 100 is able to accurately detect concentration of hydrogen as high as 10% in air. Even higher concentrations of hydrogen are detectable using the hydrogen gas sensor 100 of the present invention.

While the affinity of the nanocluster film 106 towards hydrogen is reduced, the selectively towards hydrogen is increased significantly. Thus, the nanocluster film 106 of the present invention is not prone to be rendered defunct upon exposure to such other gases as sulphur-dioxide, methane, and so on.

In yet another advantageous feature of the present invention, the nanocluster film 106 of the present invention exhibits a linear relationship between the response signal for a constant voltage power supply and hydrogen concentration in the ambient air. Thus, the hydrogen gas sensor 100 greatly simplifies calibration process, which in turn, facilitates mass scale production and practical use of such hydrogen gas sensors in various applications.

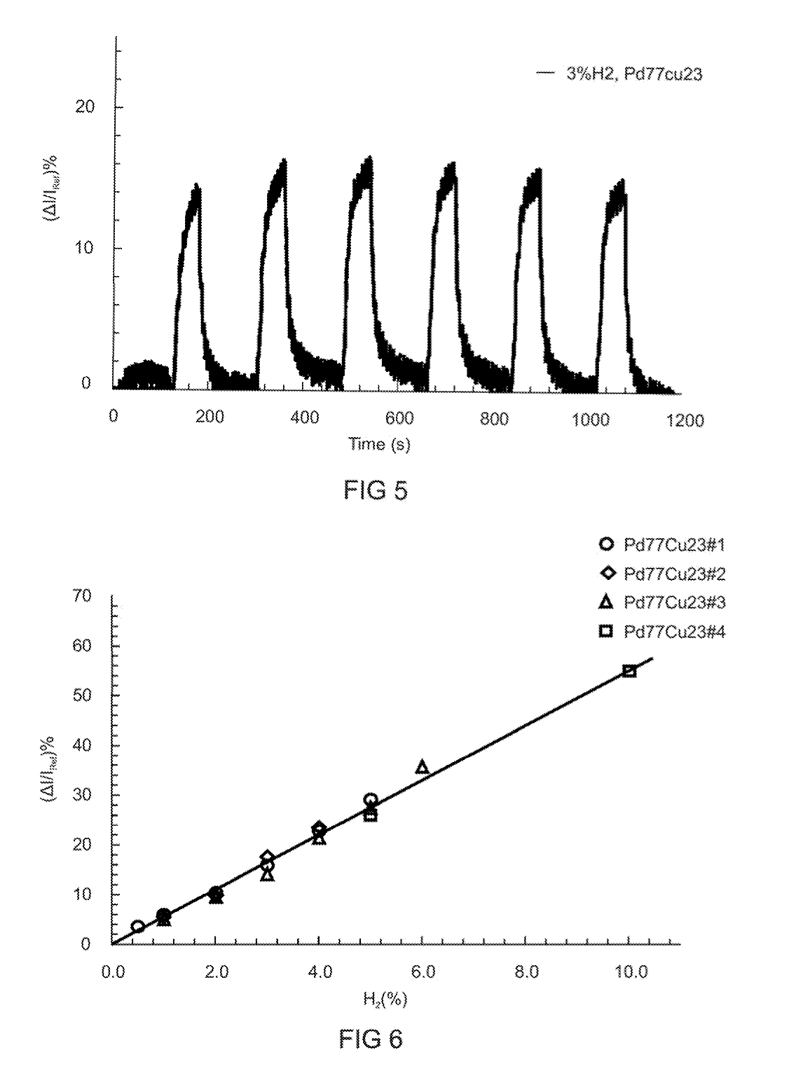

Referring now to FIG. 5, variation of response signal during measurement in a hydrogen gas sensor on repeated exposure to same hydrogen concentration is illustrated in accordance with the present invention.

The adjoining figure essentially depicts the repeatability of measurement using the hydrogen gas sensor 100 of the present invention. As can be seen in the adjoining figure, a repeatable response signal is measured across the nanocluster film 106 of the hydrogen gas sensor 100 upon exposure to a fixed concentration of hydrogen of 3%.

Furthermore, the response signal reverts to steady state once hydrogen is flushed from the ambient air. Thus, the hydrogen gas sensor 100 of the present invention can be repeatedly used for measuring hydrogen concentration and automatically recovers to be ready for performing next measurement cycle without requiring heating beyond ambient temperature.

Referring now to FIG. 6, variation of response signal during measurement in different hydrogen gas sensors on exposure to different hydrogen concentrations is illustrated in accordance with the present invention.

The adjoining figure essentially depicts reproducibility of hydrogen gas sensor 100 using the techniques of the present invention. The dependence of the electrical current as a function of hydrogen concentration for four different hydrogen gas sensors 100 is depicted. As can be readily seen, there is a linear relationship between the electrical current and hydrogen concentration, and identical slope of the relation between the response signal and hydrogen concentration for the four sensors. Therefore, the sensing properties of the four hydrogen gas sensors 100 are identical, and the fabrication process, as described herein, is a reproducible process.

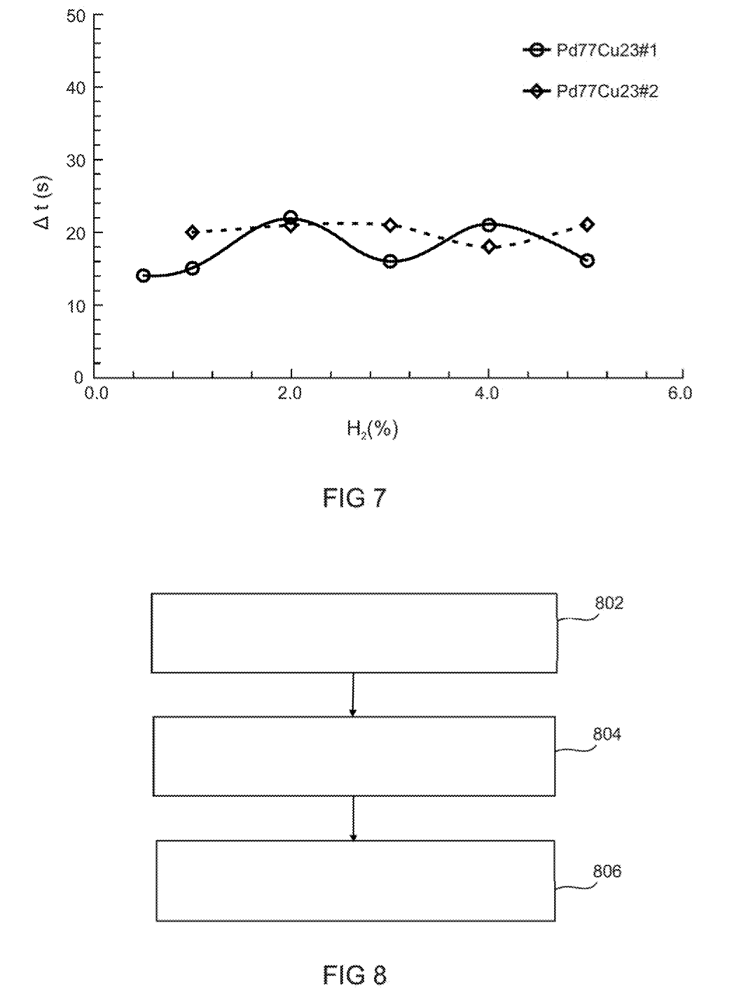

Referring now to FIG. 7, variation of response time in different hydrogen gas sensors on exposure to different hydrogen concentrations is depicted in accordance with the present invention.

The response time (or measurement time) is defined as the time needed for the response signal to increase to 90% of the maximum value. As can be seen from the adjoining figure, the hydrogen gas sensors 100 exhibit a substantially constant response time over the different hydrogen concentrations. The average response time of the hydrogen gas sensors 100 of the present invention is in the range of 18.6±2.9 s, which is a satisfactorily fast response time for various practical applications.

It should be noted that the response signal depicted in FIGS. 4(4A, 4B) through FIG. 7 have been generated using a constant voltage power supply 108 of 100 mV. If desired, the magnitude of response signal may be suitably altered by altering the power supply 108 in a required manner.

Referring now to FIG. 8, a method for fabricating a hydrogen gas sensor is illustrated in accordance with an exemplary embodiment of the present invention.

It should be noted that various steps involved in fabrication of the hydrogen gas sensor 100 have already been explained in detail in conjunction with the preceding figures. However, the method steps are being summarized below for sake of completion.

At step 802, an insulating substrate is provided and a pair of electrical electrodes is deposited thereon.

At step 804, nanoclusters of palladium-copper are generated using sputtering and inert-gas condensation techniques such that palladium ranges from about 76 percent to about 78 percent and copper ranges from about 22 percent to about 24 percent.

At step 806, a nanocluster film is deposited intermediate said electrical electrodes, wherein said nanocluster film comprises said nanoclusters of palladium-copper before the percolation threshold.

As stated earlier, detailed considerations involved at each step have already been explained in conjunction with the preceding figures.

Thus, the present invention provides a hydrogen gas sensor that simple calibration, enhanced range of operation, reduced power consumption, high selectivity, and reduced response time.

The hydrogen gas sensor of the present invention can be used in diverse applications across a range of industries. Such applications include, among others, hydrogen fuel production, hydrogen fuel cell production, petroleum refining, safety detectors, control detectors, laboratory analysis, heat treatment of metals, and basic chemical and gas analysis.

The hydrogen gas sensor of the present invention provides several advantages over those available in the state of the art.

The hydrogen gas sensor of the present invention exhibits a linear relationship between a response signal and hydrogen concentration, consequently, it is easy to calibrate.

The hydrogen gas sensor of the present invention is able to sense higher concentrations of hydrogen gas relative to conventional hydrogen gas sensors. Thus, the present invention provides hydrogen gas sensors with enhanced range of operation. The hydrogen adsorbed through the nanocluster film of the present invention is automatically released when the ambient air is devoid of hydrogen and hence, the present invention facilitates reduced power consumption.

Further, the hydrogen gas sensor of the present invention exhibits high selectively to adsorb hydrogen at relevant adsorption sites within the lattice structure of the nanocluster film and hence, is resistive to poisoning by other gaseous species.

The response time of the hydrogen gas sensor of the present invention is sufficiently low to address all practical sensing applications. The hydrogen gas sensor of the present invention exhibits desired repeatability and reproducibility properties.

While the present invention has been described in detail with reference to certain embodiments, it should be appreciated that the present invention is not limited to those embodiments. In view of the present disclosure, many modifications and variations would present themselves, to those of skill in the art without departing from the scope of various embodiments of the present invention, as described herein. The scope of the present invention is, therefore, indicated by the following claims rather than by the foregoing description. All changes, modifications, and variations coming within the meaning and range of equivalency of the claims are to be considered within their scope.

What is claimed is:

1. A method for fabricating a hydrogen gas sensor, said method comprising:
providing an insulating substrate and a pair of electrical electrodes deposited thereon,
generating nanoclusters of palladium-copper using sputtering and inert-gas condensation techniques such that palladium ranges from about 76 percent to about 78 percent and copper ranges from about 22 percent to about 24 percent, and
depositing a nanocluster film intermediate said electrical electrodes, wherein said nanocluster film comprises said nanoclusters of palladium-copper.

2. The method according to claim 1, wherein said electrical electrodes are inter-digitated electrodes, wherein separation between each pair of fingers is between 20 and 40 microns.

3. The method according to claim 1, wherein said nanoclusters within said nanocluster film have an average diameter in the range of 4 nm to 14 nm.

4. The method according to claim 1, wherein deposition of said nanocluster film comprises monitoring a signal-to-noise ratio of an electrical current established through said nanocluster film using an external power supply connected across said electrical electrodes and effecting deposition to nanoclusters till a predefined signal-to-noise ratio is achieved in said electrical current.

5. The method according to claim 1, wherein said nanocluster film is configured to be substantially near a percolation threshold thereof such that said nanocluster film is substantially non-conductive in absence of hydrogen, and further such that said nanocluster film is substantially conductive in presence of hydrogen.

6. The method according to claim 5, wherein electrical conductivity of said nanocluster film is linearly proportional to concentration of hydrogen in ambient air surrounding said nanocluster film.

7. A hydrogen gas sensor, said hydrogen gas sensor comprising:
an insulating substrate and a pair of electrical electrodes deposited thereon, and
a nanocluster film intermediate said electrical electrodes, wherein said nanocluster film comprises nanoclusters of palladium and copper generated using sputtering and inert-gas condensation techniques such that palladium ranges from about 76 percent to about 78 percent and copper ranges from about 22 percent to about 24 percent.

8. The sensor according to claim 7, wherein said electrical electrodes are inter-digitated electrodes, wherein separation between each pair of fingers is between 20 and 40 microns.

9. The sensor according to claim 7, wherein said nanoclusters within said nanocluster film have an average diameter in the range of 4 nm to 14 nm.

10. The sensor according to claim 7, wherein said nanocluster film is configured to be substantially near a percolation threshold thereof such that said nanocluster film is substantially non-conductive in absence of hydrogen, and further such that said nanocluster film is substantially conductive in presence of hydrogen.

11. The sensor according to claim 5, wherein electrical conductivity of said nanocluster film is linearly proportional to concentration of hydrogen in ambient air surrounding said nanocluster film.
